(19) United States
(12) Patent Application Publication
(10) Pub. No.: US 2016/0029882 A1
(43) Pub. Date: Feb. 4, 2016

(54) VAGINAL SPECULUM

(71) Applicant: NEW YORK UNIVERSITY, New York, NY (US)

(72) Inventor: Bruce K. Young, New York, NY (US)

(21) Appl. No.: 14/783,530

(22) PCT Filed: Jun. 11, 2014
(86) PCT No.: PCT/US2014/041843

§ 371 Date: Oct. 9, 2015

Related U.S. Application Data
(60) Provisional application No. 61/833,529, filed on Jun. 11, 2013.

(51) Int. Cl. A61B 1/303; A61B 1/32

(52) U.S. Cl. CPC A61B 1/303; A61B 1/32; A61B 2017/345

(57) ABSTRACT

A vaginal speculum comprising a supporting base oriented generally vertically. A blade extends from the base and the angle between the blade and the base is adjustable. A locking system has an unlocked position allowing the blade to be angularly moved with respect to the base and a locked position retaining the blade at that desired angle. The vaginal speculum may also have two blades, a posterior blade having the angular adjustment and an anterior blade adjustably affixed to the base such that the space between the blades may be selectively adjusted by the physician. One or both of the blades may have a plastic sleeves slidably affixed thereto. Preferably, there is a plurality of sleeves of different lengths and/or widths that can be selected and attached to the blades depending on the particular patient. That selection can be made from a kit readily available to the physician.

### BACKGROUND OF THE INVENTION

The present invention relates to a speculum for carrying out vaginal surgery, and, more particularly, to a vaginal speculum that has an angular adjustment of the blade angle as well as an adjustable blade length.

The performance of any vaginal surgery, such as dilation and curettage, hysterectomy, colporrhaphy etc require the use of a vaginal speculum. At the present, the usual vaginal specula are weighted to be self-retaining and have a fixed blade at a fixed angle.

There are two basic designs with different shapes, with some variation in length and shape of the fixed blade, however, no one speculum design fits all patients and the surgeon may ask for several different specula until one is found suitable for a specific patient, which may simply not always be possible.

The process imposes a delay to the operation where the surgeon or staff are searching for the proper speculum and often results in choosing a speculum where the weight is often too large and rests against the operating table, causing the speculum to retract poorly and slip out of the patient and fall off the patient

It would therefore be advantageous to have a vaginal speculum that was adjustable within certain parameters, that is, a vaginal speculum where the length of the blade could be selectively adjusted and where the angle of that blade was adjustable by the surgeon so that the same vaginal speculum could be used on a variety of patients.

### SUMMARY OF THE INVENTION

Accordingly, the present invention relates to a vaginal speculum that can be adjusted, both angularly as well as length-wise, in order to give the physician flexibility in operating on a patient quickly and without the need for a plurality of different specula on hand.

With the present vaginal speculum, there is a base that is oriented generally vertically and which has a blade that extends outwardly therefrom in generally a horizontal orientation. A plurality of sleeves are available that, in one exemplary embodiment, have slots such that each of the sleeves can be attached to the blade by simply inserting the blade into a slot in a sleeve. In this manner, any one of the plurality of sleeves can be selected by the physician and affixed to the blade.

Different sleeves are available having different lengths and widths such that the physician can physically examine the particular patient and then select and install the sleeve having the length and/or width that is best suited to that particular patient.

As a further feature of the present invention, the blade is angularly adjustable with respect to the base such that the physician can set the blade to a specific desired angle extending from the base and lock the blade into that angular orientation.

In a further embodiment, the vaginal speculum may have two blades, that is, there is a posterior blade and an anterior blade, both extending outwardly and generally aligned with and spaced apart from each other. In the exemplary embodiment, the anterior blade may be adjustable affixed by a sliding manner to the base to vary the space between the posterior and anterior blades and be completely detachable from the base. As such, the user can slide the anterior blade along the base to the desired location and lock the anterior blade to the base at that position, thereby establishing the desired spacing between the anterior and posterior blades.

The anterior blade may be affixed to the base by means of a mechanical linkage that includes an intermediate section having a loop, such as a C-shape loop, to form an open interior area to allow an instrument to pass through that open area for use on the patient. The vaginal speculum can be used with only the posterior blade, or with both anterior and posterior blades as needed. The sleeves can be interchangeable so as to be slipped onto both the anterior and posterior blades and have different lengths and/or widths and can be comprised of a plastic that is readily disposable.

These and other features and advantages of the present invention will become more readily apparent during the following detailed description taken in conjunction with the drawings herein.

### BRIEF DESCRIPTION OF THE DRAWINGS

FIG. 1 is a top view of the vaginal speculum of the present invention.

FIG. 2 is a rear view of the long, vertical weighted base of the vaginal speculum of FIG. 1;

FIG. 3 is a side view of the vaginal speculum of FIG. 1;

FIG. 4 is a perspective view of a sleeve of the present invention;

FIG. 5 is a side view of the sleeve of FIG. 4;

FIG. 6 is a side view of an alternative embodiment of a vaginal speculum incorporating the present invention and having a posterior blade and an anterior blade;

FIG. 7 is a rear view of the embodiment of FIG. 6;

FIG. 8 is a top view of the embodiment of FIG. 6;

FIG. 9 is an end view illustrating an alternate embodiment of a sleeve usable with the present invention;

FIGS. 10A and 10B are end views of a still further alternative embodiment illustrating a sleeve and blade, respectively, usable with the present invention; and

FIG. 11 is a schematic view of a kit containing a plurality of sleeves useable with a vaginal speculum of the present invention.

### DETAILED DESCRIPTION OF THE INVENTION

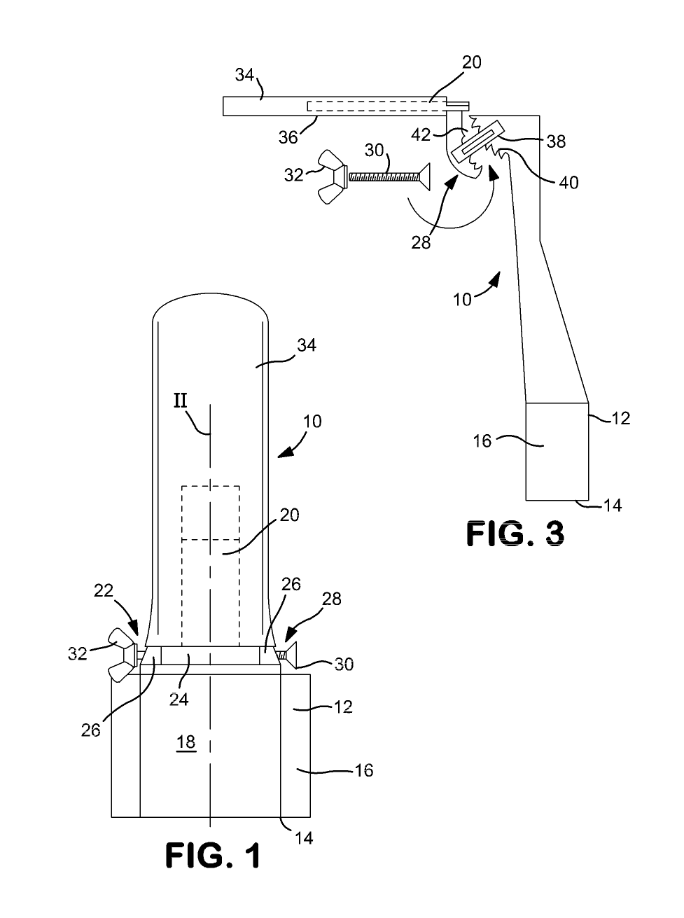

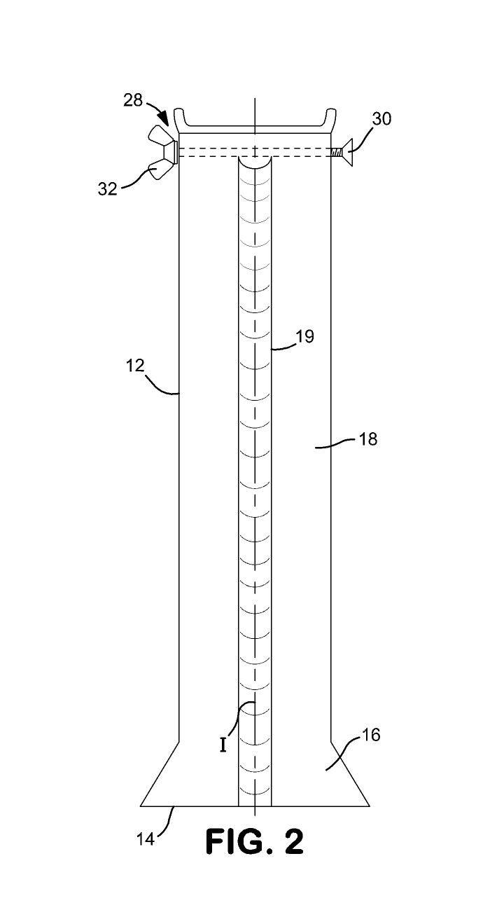

Referring now to FIGS. 1 and 2, there is shown, a top view of the vaginal speculum 10 of the present invention and a rear view of the base 12 of the vaginal speculum 10. As can be seen, the base 12 of the vaginal speculum 10 has a bottom surface 14 that is that is adapted to be positioned on a generally planar surface or hanging free. The base 12 is comprised of a base portion 16 and a generally rectangular upper portion 18. The base portion 16 is preferably heavier at its bottom such that the base portion 16 can taper outwardly toward the bottom surface 14, that is, the lateral cross section increases in the direction toward the bottom surface 14 of the base portion 16 such that the base portion 16 is weighted at the bottom for stability.

In an exemplary embodiment, the base portion 16 may be triangular in shape or may be another geometrical configuration, such as square or rectangular in horizontal cross section and a groove 19 may be provided that extends through the upper portion 18 and the base portion 16 to allow for drainage of fluids.

The base 12 is weighted so as to add stability to the vaginal speculum 10 and may be comprised of a metal, such as stainless steel, however, other materials are suitable for construction of the base 12, including, but not limited to, a high density plastic. As can be seen, specifically in FIG. 2, the longitudinal axis I of the base 12 is generally vertical when the vaginal speculum 10 is being used in connection with a patient.

Extending outwardly from the base 12 is a blade 20 that is rotatably affixed to the base 12. By that rotational affixation, the blade 20 can be moved from a position where it extends outwardly at about a right angle from the longitudinal axis I of the base 10, that is, the blade 20 can have a longitudinal axis II that is about 90 degrees from the longitudinal axis I of the base 12.

As can be seen, particularly in FIG. 1, the blade 20 is rotatable affixed to the base 12 such that the blade 20 can be rotated with respect to the base 12 to assume an angle from the base 12 that is desired by the physician using the vaginal speculum 10. In particular, the mode of affixing the blade 20 to the base 12 can be through the use of a hinge 22 with the blade 20 having a hub 24 that is sandwiched between ears 26 formed on the base 12 such that the blade 20 can be rotatable with respect to the base 12.

A locking mechanism 28 can be present in order to control the movement of the blade 12, that is, one locking mechanism that is illustrated in the exemplary embodiment is by means of a screw 30 that passes through the ears 26 and hub 24 with a threaded device, such as a wing nut 32 threadedly engaged to the free end of the screw 30.

As such the locking mechanism can have a locked position wherein the wing nut 32 is forcefully tightened along the screw 30 to pinch the hub 24 between the ears 26 to prevent the movement of the blade 20 and an unlocked position where the wing nut 32 is loosened so as to allow the blade 20 to rotate with respect to the base 12.

Accordingly, with the locking mechanism, the physician can move the locking mechanism to the unlocked position, freely move the blade 20 to the desired angular orientation based on the position and anatomy of the patient, and then lock the blade 20 in that position by moving the locking mechanism to its locked position.

Turning then to FIG. 3, taken along with FIG. 1, there is a side view of the vaginal speculum 10 of the present invention and there can be seen a sleeve 34 that is affixed to the blade 20. The sleeve 34 can be comprised of a plastic material and have a slot 36 that corresponds to the dimensions of the blade 20 such that the sleeve 34 can simply be affixed to the blade 20 by sliding the blade 20 into the slot 36, thereby affixing the sleeve 34 to the blade 20.

Further in FIG. 3, there can be seen a locking mechanism wherein there is a sliding lock 38 positioned intermediate the base 12 and the blade 20 that, again can be a tightened by means of a screw 30 and wing nut 32. In the exemplary embodiment of FIG. 3, there may be grooves acting as teeth to further prevent the movement of the blade 20 when the locking mechanism is in its locked position

As can be seen, there are grooves serving as base teeth 40 and blade teeth 42 that mesh together when the blade 20 is located in the desired angular orientation such that any relative movement between the base 12 and the blade 20 is prevented in a positive manner.

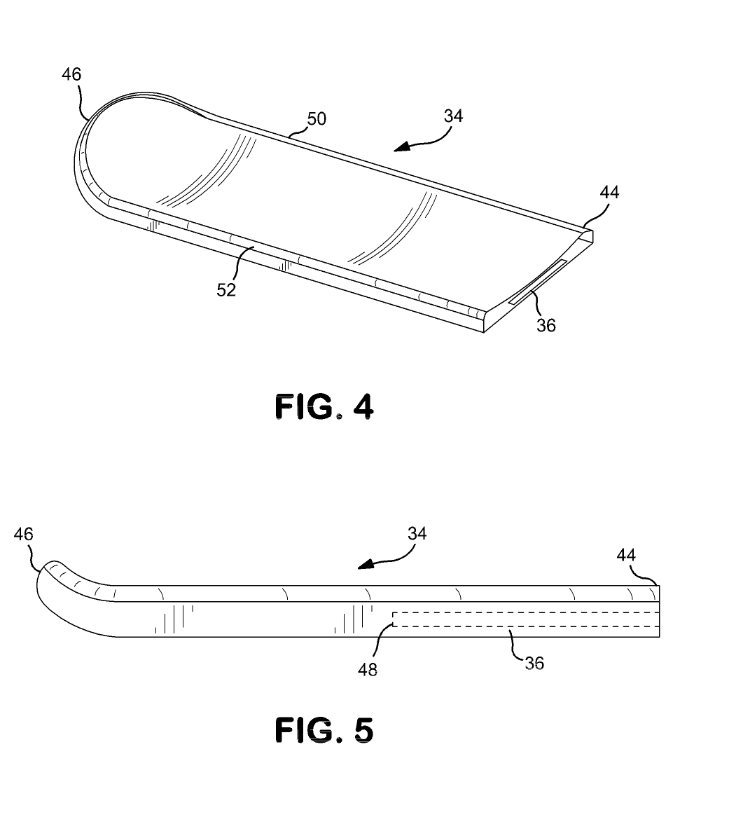

Turning then to FIGS. 4 and 5, there is a perspective view and a side view, respectively, of a sleeve 34 constructed in accordance with the present invention. As can be seen, the sleeve 34 is an elongated configuration having a proximal end 44 and a distal end 46. The slot 36 can be seen that extends from the proximal end 44 into the sleeve 34 where it ends at a wall 48.

The length of the slot 34 is, of course, determined by the length of the blade 20 (FIG. 1) and the internal dimensions of the slot 34 are predetermined so as to fit tightly over the blade 20 while still being removable therefrom In an exemplary embodiment, the overall width of the sleeve 34 is from about 2 cm to about 4 cm with the lengths from about 4 cm to about 15 cm in separate sizes, that is, 2 cm×4 cm, 3 cm×6 cm, to 5 cm to 15 cm in various sizes

The lateral edges 50, 52 of the sleeve 34 are upwardly curved and the distal end 46 is also lightly curved upwardly to facilitate the use of the sleeve 34 in carrying out a procedure on the patient.

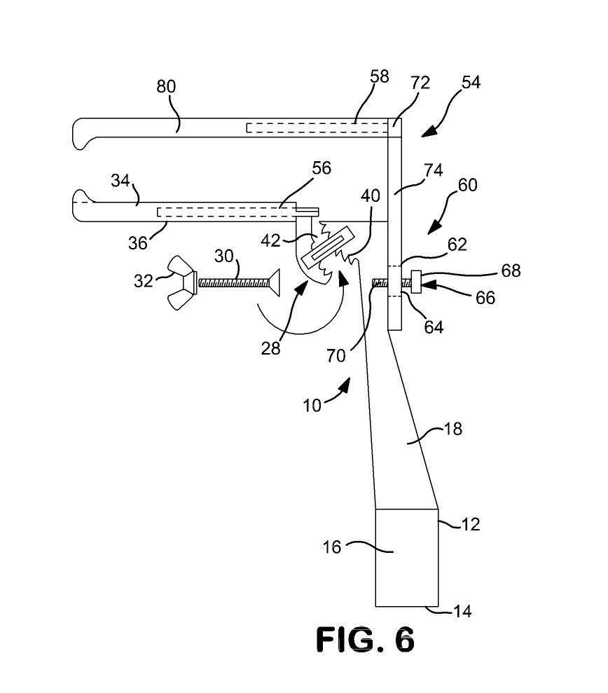

Turning then to FIG. 6, taken along with FIGS. 1 and 2, there is a side view of an alternative, exemplary embodiment of the vaginal speculum 54 constructed in accordance with the present invention. As can be seen, the vaginal speculum 54 has two blades, that is, there is a posterior blade 56 and an anterior blade 58. Taking first, the posterior blade 56, the mounting and function of the posterior blade 56 is basically the same as is shown and described in FIG. 1 and the same identification numbers have been used as in FIG. 1.

In brief, there is a base 12 of the vaginal speculum 54 having a bottom surface 14 positioned on a generally planar surface or hanging free and includes a base portion 16 and a generally rectangular upper portion 18.

Extending outwardly from the base 12 is the posterior blade 56 that is rotatable affixed to the base 12 in the manner described with respect to the embodiment of FIGS. 1-3. Again, the angular orientation of the posterior blade 56 with respect to the base 12 is determined and controlled by use of a hinge 22 with the blade 56 having a hub 24 that is sandwiched between ears 26 formed on the base 12 such that the blade 56 can be rotatable with respect to the base 12.

A locking mechanism 28 controls the movement of the blade 12 and may be means of a screw 30 that passes through the ears 26 and hub 24 with a threaded device, such as a wing nut 32 threadedly engaged to the free end of the screw 30.

Accordingly, by the construction of the hinge 22 and locking mechanism, the physician can move the posterior blade 56 to a desired angular position with respect to the base 12 and simply lock the blade 56 in that position

As to the anterior blade 58, it too is affixed to the base 12 and, in the exemplary embodiment of FIG. 6, there is a mechanical linkage 60 that affixes the anterior blade 58 to the base 12. The mechanical linkage 60 will be further later explained , however, the mechanical linkage 60 is used to mount the anterior blade 58 to the base 12 and includes a lower flange section 62 that slides vertically along the base 12. The lower flange section 62 has an elongated slot 64 formed therein and a screw 66 having a large head 68 can pass through the elongated slot 64 to be screwed into a threaded opening 70 in the base 12.

As such, the location of the lower flange section 62, and thus the location of the anterior blade 58 can be adjusted with respect to the base 12 by loosening the screw 66, vertically moving the lower flange section 62 to the desired location and then tightening the screw 66 in the threaded opening 70 to firmly press the screw head 68 against the lower flange section 62 to affix the lower flange section 62 at the desired location. As can be seen, by adjusting the location of the lower flange section 62 with respect to the base 12, the space between the poosterior blade 56 and the anterior blade 58 can be adjusted according to the desire of the user.

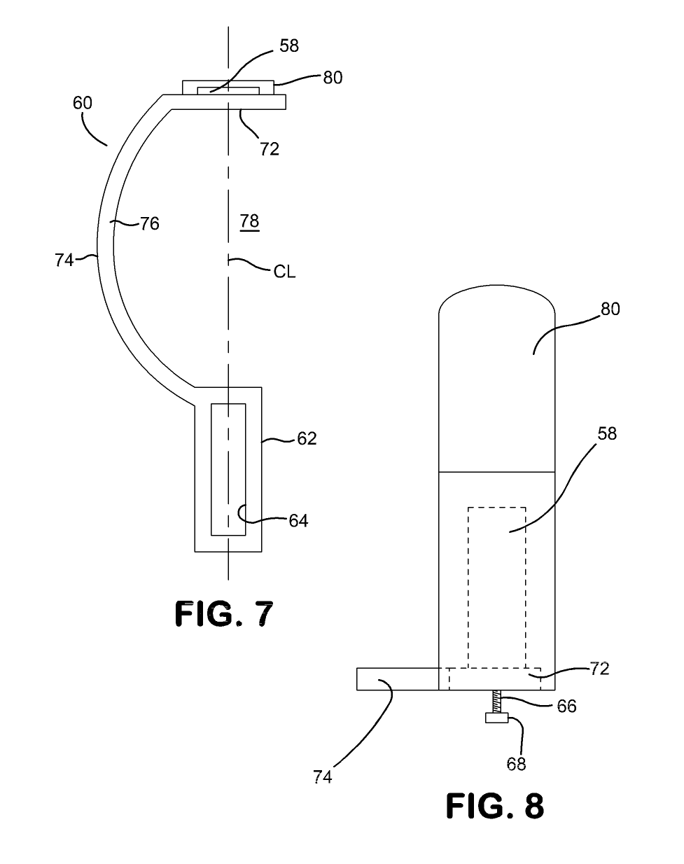

Turning to FIG. 7, taken along with FIG. 6, the mechanical linkage 60 can be seen more clearly and includes the lower flange section 62, an upper arm 72 and an intermediate section 74. The anterior blade 58 extends outwardly from the upper arm 72 and the main body 76 of the intermediate section 74 is designed to be displaced outwardly from the centerline CL of the lower flange section 62 As such there is an open area 78 that is created between the lower flange section 62 and the upper arm 72 that allows the physician to insert and manipulate an instrument on the patient without interference from the mechanical linkage 60.

In the exemplary embodiment of FIG. 7, it can be seen that the intermediate section 74 is a generally C-shaped curved configuration; however, the intermediate section 74 may be other shapes providing there remains the open area 78 to facilitate the introduction of a medical instrument as previously described. In addition, in the illustration of FIG. 7, the open area 78 is open to the right, as viewed in that Figure which accommodates a right handed physician, however, the open area 78 may also extend to the left to accommodate a left handed physician.

A sleeve 80 is slipped over the distal end of the anterior blade 58 and the sleeve 80 is held to the anterior blade 58 by a friction fit to the anterior blade 58 in a manner similar to or the same as the sleeve 34 that is affixed to the posterior blade 56. Both sleeves may the same material and construction and preferable both sleeves are constructed of a plastic material so as to be disposable after a single use.

Again, with the anterior blade 58 and posterior blade 56, the sleeves 34 and 80 can be selected from a plurality of sleeves having different lengths and/or widths so that the physician can select and use the appropriate sleeve for the particular patient. For efficiency, the sleeves 34 and 80 may be interchangeable.

Turning then to FIG. 8, there is shown a top view of the embodiment of FIG. 6 illustrating the sleeve 80 slipped onto the anterior blade 58. As described, the sleeve 80 has a slot that is formed therein so the as the sleeve 80 is slipped onto the anterior blade 58 such that the anterior blade 58 enters the slot in a friction fit to allow the sleeve 80 to be affixed to the anterior blade 58.

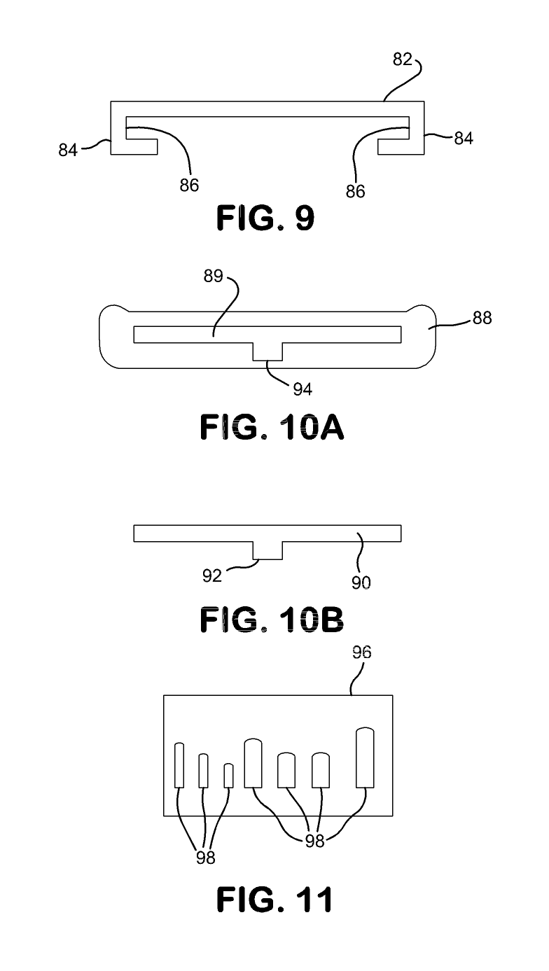

In FIG. 9, there is shown an alternative sleeve 82 that is usable with the present invention. In particular, it can be seen that the sleeve 82 has a pair of downwardly directed edges 84 so as to form lateral slots 86 that are dimensioned so as to allow the sleeve 82 to be slid onto a blade by sandwiching the blade between the slots 86 to slidingly securing the sleeve 82 to a blade. With this embodiment, it can be seen that the blade does not need to have a slot formed therein.

In addition, in FIG. 10A and 10B, there is shown a still further alternative exemplary embodiment of the present invention wherein a sleeve 88 is slipped onto a blade 90. In FIG. 10B, the blade 90 has a projection 92 extending outwardly therefrom that interfits into a corresponding longitudinal slot 94 formed in the slot 89 of sleeve 88. With this embodiment, the interfitting of the projection 92 of the blade 90 provides lateral stability to the affixation of the blade 90 to the sleeve 88. While only one projection 92 is shown interfitting into one longitudinal slot 94, it can be seen that there may be a plurality of projections of differing sizes and shapes that interfit onto a corresponding slot or slots in the sleeve.

With the present vaginal speculum 10, therefore, the physician can have a plurality of sleeves 34 (FIG. 1) having different lengths and widths and select the one prior to the operation that best suits the anatomy of the patient after an on site examination of the patient. In particular, the sleeves may be provided in lengths of from 5 cm to about 15 cm in 1 cm increments.

With a plurality of sleeves available to the physician, there can be a kit 96 shown in FIG. 11 that is located near the physician so that the physician can select the most appropriate sleeve 98 having the desired length or width for each patient such that the vaginal speculum is adaptable to different patients without the need to delay an operation in order to locate a vaginal speculum of the correct length.

As such, each kit 96 would contain different sleeves 98 of varying lengths and/or widths for selection by the physician after an initial examination of the patient and such kit 96 would be readily accessible to the physician so that the physician could select and attach the desired sleeve 98 to a blade depending on the particular patient.

Also as a variant of the sleeves 98, the proximal ends of the sleeves 98 may be open so as to receive a blade therein or may be closed such as where a affixation of the sleeves to a blade is in accordance with FIG. 9 where there is no need for a slot in a sleeve.

Those skilled in the art will readily recognize numerous adaptations and modifications which can be made to the vaginal speculum of the present invention which will result in an vaginal speculum and method of using the vaginal speculum, yet all of which will fall within the scope and spirit of the present invention as defined in the following claims Accordingly, the invention is to be limited only by the following claims and their equivalents.

What is claimed is:

1. A vaginal speculum comprising a base, a blade affixed to the base and extending outwardly therefrom, the blade being angularly adjustable with respect to the base, at least one sleeve removably affixed to the blade and extending outwardly therefrom.

2. The vaginal speculum of claim 1 wherein the base has a bottom and wherein the base has an increasing cross section in the direction towards the bottom, the base having a generally vertical longitudinal axis.

3. The vaginal speculum of claim 1 wherein the blade extends along a longitudinal axis at about a 90 degree angle with respect to the longitudinal axis of the base and the 90 degree angle varies as the blade is angularly adjusted with respect to the base.

4. The vaginal speculum of claim 1 wherein the blade is angularly adjustable by means of a plurality of intermeshing teeth so as to allow the blade to move to different angles with respect to the base and a locking mechanism to lock the blade in the desired angular configuration.

5. The vaginal speculum of claim 1 wherein the intermeshing teeth comprise a first set of teeth fixed in position with respect to the base and a second set off teeth fixed in position with respect to the blade and the teeth are selectively meshed and unmeshed.

6. The vaginal speculum of claim 4 wherein the locking mechanism comprises a screw and nut adapted to tighten the blade in the desired angular position with the first and second sets of teeth intermeshed.

7. The vaginal speculum of claim 1 wherein the sleeve has lateral downwardly directed edges forming lateral slots formed therein and the blade is interfitted into the lateral slots.

8. The vaginal speculum of claim 1 wherein the blade has at least one projection extending outwardly therefore and the sleeve has at least one longitudinal slot wherein the at least one projection interfits into the at least one slow when the sleeve is affixed to the blade.

9. The vaginal speculum of claim 1 wherein the at least one sleeve comprises a plurality of sleeves, each being removable attachable to the blade, the plurality of sleeves including sleeves having different lengths.

10. A method of using a vaginal speculum on a patient, the method comprising the steps of:
providing a vaginal speculum having a base and having a blade extending therefrom,
providing at least one sleeve,
affixing the at least one sleeve to the blade by sliding the sleeve over the blade in a friction fit
adjusting the angle of the blade with respect to the base,

11. The method of claim 10 wherein the at least one sleeve comprise a plurality of sleeves, each of which are individually attachable to the blade and further including the step of selecting the desired length of a sleeve and attaching the selected sleeve to the blade.

12. The method of claim 10 wherein the step of affixing the at least one sleeve comprises sliding the sleeve over the blade to create a friction fit therewith.

13. A kit for use by a physician undertaking a gynecological procedure, the kit comprising a plurality of disposable sleeves of differing lengths, each sleeve having a slot adapted to snugly interfit with a blade of a vaginal speculum for carrying out the procedure.

14. A vaginal speculum comprising a base, a posterior blade affixed to the base and extending outwardly therefrom, the posterior blade being angularly adjustable with respect to the base, a first sleeve removably affixed to the posterior blade and extending outwardly therefrom, an anterior blade affixed to the base and extending outwardly therefrom, the anterior blade being oriented generally in alignment with the posterior blade, the anterior blade having a second sleeve removably affixed thereto.

15. The vaginal speculum as defined in claim 14 wherein the first and second sleeves are interchangeable.

16. The vaginal speculum of claim 14 wherein the first and second sleeves are comprised of a disposable plastic material

17. The vaginal speculum of claim 14 wherein the blade is affixed to the base by a mechanical linkage that is adjustably affixed to the base to selectively set the spacing between the posterior blade and the anterior blade.

18. The vaginal speculum of claim 17 wherein the mechanical linkage has a lower flange section that is adjustably affixed to the base and an intermediate section located between the lower flange section and the anterior blade.

19. The vaginal speculum of claim 17 wherein the lower flange section has an elongated opening and a screw is provided to pass through the elongated opening to tighten the lower flange section to the base at a desired location.

20. The vaginal speculum of claim 18 wherein the intermediate section is a C-shaped configuration with an open area to allow an instrument to be passed through the open area for use on a patient.
